(19) United States
(12) Patent Application Publication
(10) Pub. No.: US 2024/0410904 A1
(43) Pub. Date: Dec. 12, 2024

(54) NON-INVASIVE MONITORING OF LIVER DISEASE TREATMENT

(71) Applicant: INVENTIVA, DAIX (FR)

(72) Inventors: Pierre BROQUA, Daix (FR); Philippe HUOT-MARCHAND, Daix (FR); Lucile DZEN, Daix (FR); Jérôme BOURSIER, Brissac Loire Aubance (FR); Marine ROUX, Feneu (FR); Hugo HERVÉ, Plérin (FR)

(73) Assignee: INVENTIVA, DAIX (FR)

(21) Appl. No.: 18/735,392

(22) Filed: Jun. 6, 2024

Related U.S. Application Data
(63) Continuation of application No. PCT/IB2023/000353, filed on Jun. 6, 2023.

(51) Int. Cl. G01N 33/68; A61K 31/426; G16H 20/10

(52) U.S. Cl. CPC G01N 33/6893; A61K 31/426; G16H 20/10; G01N 2333/47; G01N 2333/91188; G01N 2400/10; G01N 2800/52

(57) ABSTRACT

The present disclosure relates to a method of assessing the effectiveness of a treatment with lanifibranor in a patient with a liver disease, the method comprising:
a) in vitro measuring levels of a combination of biomarkers consisting of CK18M65, hyaluronic acid, fructosamine and ALT in a biological sample from the patient;
b) assessing the effectiveness of the treatment with lanifibranor as a function of the levels measured in step a).

### FIELD OF THE INVENTION

The present disclosure relates to a method of assessing the effectiveness of a treatment with the investigational drug lanifibranor in a patient with a liver disease. The present disclosure also relates to a method of treating a liver disease comprising a step of assessing the effectiveness of the lanifibranor treatment.

### BACKGROUND OF THE INVENTION

Nonalcoholic steatohepatitis (NASH) is a major cause of chronic liver disease. Its diagnosis and characterization currently rely on histological investigations and liver biopsies are still required for the diagnosis and assessment of treatment response. However, considering the invasive nature of liver biopsies as well as shortcomings of histological evaluation such as high sampling variability, poor inter-observer evaluation and potential complications there is an urgent need for alternative diagnostic methods. The identification of biomarker signatures for non-invasive assessment of the histological response would be an important step to overcome the shortcomings of currently used diagnostic methods and facilitate NASH therapies.

The pan-PPAR (peroxisome proliferator-activated receptor) agonist lanifibranor is a promising investigational compound that modulates key metabolic, inflammatory, and fibrogenic pathways and has demonstrated therapeutic efficacy on both NASH resolution and fibrosis improvement in the phase 2b NATIVE trial. The study revealed encouraging results, with the percentage of patients with active NASH who had a decrease of at least 2 points in the Steatosis Activity Fibrosis (SAF)-A score without worsening of fibrosis being significantly higher after treatment with a daily dose of 1200 mg lanifibranor compared to treatment with placebo. Secondary endpoints of this study included NASH resolution and fibrosis improvement, NASH resolution without worsening of fibrosis and improvement of fibrosis without worsening of NASH, according to NASH Clinical Research Network (NASH-CRN) (Results of this study were more favorable than placebo in the lanifibranor groups.

In this context, the aim of the inventors was to identify biological signatures of histological responders among NASH patients treated with lanifibranor. The identification and development of a biomarker signature can indeed aid in assessing the response to a treatment and help identifying patients most likely to experience clinical benefit from the treatment.

### SUMMARY OF THE INVENTION

The present disclosure relates to a method of assessing the effectiveness of treatment with lanifibranor in a patient with a liver disease, the method comprising:
a) in vitro measuring levels of a combination of biomarkers consisting of CK18M65, hyaluronic acid, fructosamine and ALT in a biological sample from the patient, and
b) assessing the effectiveness of the treatment with lanifibranor as a function of the levels measured in step a).

In some embodiments, the level of CK18M65 is measured before the start of the treatment with lanifibranor.

In some embodiments, levels of hyaluronic acid, fructosamine and ALT are measured before the start of the treatment with lanifibranor and after at least 3 months of treatment with lanifibranor.

In some embodiments, the levels measured in step a) are used to obtain a score which falls within a determined or undetermined prognosis class. In some embodiments, the determined prognosis class includes a class of predicted responders to the lanifibranor treatment and a class of predicted non-responders to the lanifibranor treatment. In some embodiments, the score is obtained by combining the levels measured in step a) in a mathematical function. In some embodiments, the mathematical function is a binary logistic regression. In some embodiments, the method comprises comparing the score with a first calculated cutoff value below which no response to the lanifibranor treatment is predicted. In some embodiments, the method comprises comparing the score with a second calculated cutoff value above which a response to the lanifibranor treatment is predicted.

In some embodiments, the biological sample is a sample of biological fluid. In some embodiments, the biological fluid is blood, serum or plasma.

In some embodiments, the liver disease is non-alcoholic fatty liver disease, non-alcoholic steatohepatitis, compensated or decompensated cirrhosis, liver fibrosis, fatty liver disease, acute liver failure or acute-on-chronic liver failure.

The present disclosure also relates to a system for assessing the effectiveness of a treatment with lanifibranor in a patient with a liver disease, the system comprising:
a) means for measuring or receiving measurement data of levels of a combination of biomarkers consisting of CK18M65, hyaluronic acid, fructosamine and ALT in a biological sample from the patient; and
b) means for processing the data configured to assess the effectiveness of the treatment with lanifibranor in the patient as a function of the levels measured for the combination of biomarkers.

The present disclosure also relates to a combination of biomarkers consisting of CK18M65, hyaluronic acid, fructosamine and ALT for use in a method of assessing the effectiveness of a treatment with lanifibranor in a patient with a liver disease.

The present disclosure also relates to a method of treating a liver disease in a subject, the method comprising:
a) in vitro measuring levels of a combination of biomarkers consisting of CK18M65, hyaluronic acid, fructosamine and ALT in a biological sample from the patient before initiating a treatment;
b) daily administering an effective amount of lanifibranor to the subject for at least 3 months;
c) in vitro measuring levels of hyaluronic acid, fructosamine and ALT in a biological sample of the subject after 3 months of treatment;
d) assessing the effectiveness of the treatment with lanifibranor as a function of the levels measured in steps a) and c).
e) continuing administering an effective amount of lanifibranor to the subject for at least another 3 months provided the assessment made in step d) is predictive of a response to the lanifibranor treatment, or that no prognosis can be made.

### DESCRIPTION OF THE FIGURES

FIG. 1 shows the sensitivity, specificity, NPV and PPV as a function of an E2-score.

### DETAILED DESCRIPTION OF THE INVENTION

The present disclosure relates to a method of assessing the effectiveness of treatment with lanifibranor in a patient with a liver disease, the method comprising:
a) in vitro measuring levels of a combination of biomarkers consisting of CK18M65, hyaluronic acid, fructosamine and ALT in a biological sample from the patient, and
b) assessing the effectiveness of the treatment with lanifibranor as a function of the levels measured in step a).

In the context of the present disclosure, CK18M65 stands for cytokeratin 18 (antigen M65); ALT stands for alanine transaminase.

In the context of the present disclosure, “combination of biomarkers”, “biomarker signature” and “biomarkers signature” are used interchangeably.

As used herein “assessing the effectiveness/efficacy of a treatment” herein refers to the determination of the clinical condition of a patient subjected to a treatment. The treatment may be preventive, for example in the case of predisposition to a disease, or it may be curative, for example in the case of a diagnosed disease. The effectiveness of the treatment may for example be evaluated by determining the condition of the patient at different time intervals. The condition of the patient may notably be evaluated before the first taking of the treatment then at regular (or irregular) time intervals after this first taking (for example after each new taking of the treatment). A comparison of the condition of the patient evaluated at these different intervals may then be carried out in order to identify a potential change. The condition of the patient may be evaluated on the basis of observations and/or measurements, carried out using different tools.

As used herein, “treatment” refers to any process, action, application, therapy, or the like, wherein the patient is under aid, in particular, medical, or veterinarian aid with the object of improving the patient's condition, either directly or indirectly.

As used herein, “patient” or “subject” refers to a human individual or an animal different from a human. The patient is for example a human or an animal liable to have a liver disease or suffering from such a disease. The patient is advantageously a human being. The patient may be a child (human patient 18 years old or less) or an adult (human patient more than 18 years old). In the context of the present disclosure the terms “patient” and “subject” are used interchangeably.

As used herein, “measuring” or “measurement” is understood as quantitative characterization of a physical object or entity or a multitude (population or plurality) thereof, or their function or quantitative characterization of a physical or chemical process, comprising the assignment of a quantity, value, e.g. a numerical value or a number characteristic of the object or entity or multitude or function or process, by comparison with units and, in comparison with another object or entity or multitude or function or process. Preferably a measurement is consistent with methods known in the art or the international guidelines of metrology.

As used herein, “quantifying” or “quantification” or “quantitation” is understood herein as an assignment of a physical quantity to a physical object or entity or a multitude (population or plurality) thereof, or their function or quantitative characterization of a physical or chemical process, expressed in a numerical value or number and units, and, in comparison with another object or entity. Advantageously, “quantifying” or “quantification” is a measurement or an essential part of a measurement. The measurement has an uncertainty which may represent the random and systemic errors of the measurement procedure. The skilled person is aware of this and can handle this error in view of the measurement or quantification applied.

As used herein, lanifibranor is a pan-PPAR agonist of formula {4-[1-(1,3-benzothiazol-6-ylsulfonyl)-5-chloroindol-2-yl]butanoic acid; CAS 927961-18-0}. Advantageously, the term “lanifibranor” also includes any deuterated form of lanifibranor or any pharmaceutical salt thereof. Deuterated forms of lanifibranor can be those disclosed in international application WO2020/021215, the disclosure of which is incorporated by reference.

In some embodiments, biomarkers the level of which is measured at step a) are protein biomarkers. Advantageously, biomarkers are serum biomarkers.

In some embodiments, the expression level of CK18M65 is measured before the start of the treatment with lanifibranor.

In some embodiments, levels of hyaluronic acid, fructosamine and ALT are measured before the start of the treatment with lanifibranor and after at least 3 months of treatment with lanifibranor. Advantageously, levels of hyaluronic acid, fructosamine and ALT are measured after at least 4 months, advantageously after at least 5 months, advantageously after at least 6 months, advantageously after at least 7 months, advantageously after at least 8 months, advantageously after at least 9 months, advantageously after at least 10 months, advantageously after at least 11 months, advantageously after at least 12 months, or more of treatment with lanifibranor.

In some embodiments, the levels measured in step a) are used to obtain a score which falls within a determined or undetermined prognosis class. In some embodiments, the determined prognosis class includes a class of predicted responders to the lanifibranor treatment and a class of predicted non-responders to the lanifibranor treatment.

In some embodiments, the score is obtained by combining the levels measured in step a) in a mathematical function. In some embodiments, the mathematical function is a binary logistic regression.

In some embodiments, the method comprises comparing the score with a first calculated cutoff value below which no response to the lanifibranor treatment is predicted. In an advantageous embodiment, the first calculated cutoff value is equal to about 0.453. In some embodiments, the method comprises comparing the score with a second calculated cutoff value above which a response to the lanifibranor treatment is predicted. More particularly, when the score obtained (also hereafter “E2-score”) is above the second calculated cutoff value, resolution of NASH and no worsening of liver fibrosis can be predicted. In an advantageous embodiment, the second calculated cutoff value is equal to about 0.646.

In some embodiments, the biological sample is a sample of biological fluid. In some embodiments, the biological fluid is blood, serum or plasma. Advantageously, the biological fluid is blood.

In some embodiments, the liver disease is non-alcoholic fatty liver disease, non-alcoholic steatohepatitis, cirrhosis such as compensated or decompensated cirrhosis, liver fibrosis, fatty liver disease, acute liver failure or acute-on-chronic liver failure.

The present disclosure also relates to a system for assessing the effectiveness of a treatment with lanifibranor in a patient with a liver disease, the system comprising:
a) means for measuring or receiving measurement data of levels of a combination of biomarkers consisting of CK18M65, hyaluronic acid, fructosamine and ALT in a biological sample from the patient; and
b) means for processing the data configured to assess the effectiveness of the treatment with lanifibranor in the patient as a function of the levels measured for the combination of biomarkers.

The present disclosure also relates to a combination of biomarkers consisting of CK18M65, hyaluronic acid, fructosamine and ALT for use in a method of assessing the effectiveness of a treatment with lanifibranor in a patient with a liver disease.

In some embodiments, the liver disease is non-alcoholic fatty liver disease, non-alcoholic steatohepatitis (NASH), cirrhosis such as compensated or decompensated cirrhosis, liver fibrosis, fatty liver disease, acute liver failure or acute-on-chronic liver failure.

In some embodiments, the treatment with lanifibranor allows resolution of NASH and no worsening of liver fibrosis.

The present disclosure also relates to a method of treating a liver disease in a subject, the method comprising:
a) in vitro measuring levels of a combination of biomarkers consisting of CK18M65, hyaluronic acid, fructosamine and ALT in a biological sample from the patient before initiating a treatment;
b) daily administering an effective amount of lanifibranor to the subject for at least 3 months;
c) in vitro measuring levels of hyaluronic acid, fructosamine and ALT in a biological sample of the subject after 3 months of treatment;
d) assessing the effectiveness of the treatment with lanifibranor as a function of the levels measured in steps a) and c), whereby a response or a non-response to the lanifibranor treatment can be predicted;
e) continuing administering an effective amount of lanifibranor to the subject for at least another 3 months provided the assessment made in step d) is predictive of a response to the lanifibranor treatment, or that no diagnostic can be made.

According to embodiments that involve administering to a patient in need of treatment a (therapeutically) effective amount of lanifibranor, “therapeutically effective” or “effective amount” or “an amount effective to treat” or “pharmaceutically effective” denotes the amount of lanifibranor needed to inhibit or reverse a disease condition (e.g., to treat liver disease). Determining an effective amount specifically depends on such factors as safety and efficacy of the medicament. These factors will differ depending on other factors such as potency, relative bioavailability, patient body weight, severity of adverse side-effects and preferred mode of administration. Safety may be determined using methods well known in the art. Efficacy may be determined utilizing the same guidance. A pharmaceutically effective amount, therefore, is an amount that is deemed by the clinician to be safe, and efficacious.

Dosage may be adjusted appropriately to achieve desired level, local or systemic, depending upon the mode of administration. In the event that the response in a patient is insufficient at such doses, even higher doses (or effective higher doses by a different, more localized delivery route) may be employed to the extent that patient tolerance permits. Multiple doses per day may also be employed to achieve appropriate systemic levels of lanifibranor. Appropriate systemic levels can be determined by, for example, measurement of the patient's peak or sustained plasma level of the drug. “Dose” and “dosage” are used interchangeably herein.

In some embodiments, according to step b) of a method of treating a liver disease in a subject, lanifibranor is administered at a daily dosage of from about 400 mg to about 1,200 mg. Advantageously, lanifibranor is administered at a daily dosage of about 400 mg, advantageously at a daily dosage of about 500 mg, advantageously at a daily dosage of about 600 mg, advantageously at a daily dosage of about 700 mg, advantageously at a daily dosage of about 800 mg, advantageously at a daily dosage of about 900 mg, advantageously at a daily dosage of about 1,000 mg, advantageously at a daily dosage of about 1,100 mg, advantageously at a daily dosage of about 1,200 mg. In some embodiments, lanifibranor is administered to a patient with a meal. In some embodiments, lanifibranor is administered to a patient under fasted conditions.

In some embodiments, lanifibranor is employed for in vivo applications. Depending on the intended mode of administration in vivo, lanifibranor may be administered as a solid, semi-solid or liquid dosage form. In some embodiments, lanifibranor is administered in a solid dosage form. Exemplary solid dosage forms include tablets, capsules, stick-packs, sachets, lozenges, powders, pills, or granules. Preferred solid dosage forms include tablets, capsules and stick-packs, tablets being especially preferred. Advantageously, the lanifibranor is administered in unit dosage forms suitable for single administration of precise dosage amounts. Depending on the formulation desired, lanifibranor can be formulated into a pharmaceutical composition comprising lanifibranor and one or more pharmaceutically acceptable excipient(s). The choice of excipient(s) will to a large extent depend on factors such as the particular mode of administration, the effect of the excipient on solubility and stability, and the nature of the dosage form. Pharmaceutical compositions of the invention can be prepared by conventional methods, as described e.g. in Remington's Pharmaceutical Sciences, 19th Edition (Mack Publishing Company, 1995), incorporated herein by reference. In some embodiments, the pharmaceutically acceptable excipients include two or more of a binder, a disintegrant, a filler, a glidant, a lubricant, and a surfactant. In some embodiments, the pharmaceutically acceptable excipients include a binder, a disintegrant, a filler, a glidant, a lubricant and a surfactant.

In some embodiments, the pharmaceutical composition comprises from 200 mg to 1,200 mg of lanifibranor. Exemplary pharmaceutical compositions comprise 200 mg, 400 mg, 600 mg, 800 mg, 1,000 mg or 1,200 mg of lanifibranor.

In some embodiments, lanifibranor is administered at a daily dosage of from about 400 mg to about 1,200 mg.

In any of the embodiments described above, lanifibranor either per se or when present in a pharmaceutical composition, can be in crystalline form.

Administration during in vivo treatment may be by any routes, including oral, parenteral, intramuscular, intranasal, sublingual, intratracheal, inhalation, ocular, vaginal, and rectal.

The skilled person in the art will recognize that the route of administration varies depending on the disorder to be treated. Advantageously, lanifibranor or the pharmaceutical composition comprising lanifibranor may be administered to a patient via oral, parenteral or topical administration. In one embodiment, the pharmaceutical composition comprising lanifibranor is administered by oral administration.

In some embodiments, according to step b) of the method, lanifibranor is daily administered in an effective amount to the subject for at least 3 months, advantageously at least 4 months, advantageously at least 5 months, advantageously at least 6 months, advantageously at least 7 months, advantageously at least 8 months, advantageously at least 9 months, advantageously at least 10 months, advantageously at least 11 months, advantageously at least 12 months, or more.

In some embodiments, according to step c) of the method, levels of hyaluronic acid, fructosamine and ALT are measured in a biological sample of the subject after at least 3 months, advantageously at least 4 months, advantageously at least 5 months, advantageously at least 6 months, advantageously at least 7 months, advantageously at least 8 months, advantageously at least 9 months, advantageously at least 10 months, advantageously at least 11 months, advantageously at least 12 months, or more of treatment.

In some embodiments, the assessment in step d) is made by obtaining a score (also called E2-score) from the levels measured in steps a) and c), and comparing the score (i) with a first calculated cutoff value below which no response to the lanifibranor treatment can be predicted, and (ii) with a second calculated cutoff value above which a response to the lanifibranor treatment can be predicted. When the score obtained is below the first calculated cutoff value, no response (i.e. negative response) to the lanifibranor treatment can be predicted. When the score obtained is above the second calculated cutoff value, a response (i.e. positive response) to the lanifibranor treatment can be predicted. In particular, when the score obtained is above the second calculated cutoff value, resolution of NASH and no worsening of liver fibrosis can be predicted. When the score obtained is between the first calculated cutoff value and the second calculated cutoff value, no prognosis can be made as to the effectiveness of the lanifibranor treatment.

In an advantageous embodiment, the first calculated cutoff value is equal to about 0.453.

In an advantageous embodiment, the second calculated cutoff value is equal to about 0.646.

In some embodiments, according to step e) of the method, lanifibranor is administered in an effective amount to the subject for at least another 3 months, advantageously at least another 4 months, advantageously at least another 5 months, advantageously at least another 6 months, advantageously at least another 7 months, advantageously at least another 8 months, advantageously at least another 9 months, advantageously at least another 10 months, advantageously at least another 11 months, advantageously at least another 12 months, or more.

In some embodiments, the duration of the treatment with lanifibranor as mentioned in step e) is dependent on how long lanifibranor has been administered in step b). In some embodiments, the measurement in step c) is performed after the treatment with lanifibranor. If in step b), the treatment with lanifibranor is of 3 months, the measurement in step c) is performed after the 3 months treatment.

In some embodiments, the biological sample is a sample of biological fluid. In some embodiments, the biological fluid is blood, serum or plasma. Advantageously, the biological fluid is blood.

In some embodiments, the liver disease is non-alcoholic fatty liver disease, non-alcoholic steatohepatitis, compensated or decompensated cirrhosis, liver fibrosis, fatty liver disease, acute liver failure or acute-on-chronic liver failure.

In some embodiments, the present disclosure also includes kits for evaluating the effectiveness of the treatment with lanifibranor as well as kits for determining CK18M65, hyaluronic acid, fructosamine and ALT levels, for example, from a biological sample. As used herein, “kit” is intended to mean a package, collection, or container of materials intended to aid one in use of the assay of the invention.

Kits of the present disclosure will typically comprise one or more containers containing one or more reagents useful to practice the invention. Reagents useful to practice the invention include, but are not limited to, buffers, buffer salts, metal ions, chromogenic compounds, antibodies, enzymes, fluorescent compounds and the like. Kits of the present disclosure may comprise one or more containers containing CK18M65, hyaluronic acid, fructosamine and ALT, or other compounds that may be used as a reference standard. Kits of the invention may comprise containers containing one or more antibodies wherein the antibodies are conjugated to a detectable moiety. Detectable moieties may be any known to those skilled in the art, for example, enzymes (e.g., peroxidase, luciferase), other proteins (e.g., green fluorescent protein), optically detectable compounds (e.g., fluorophores, chromophores), members of a binding pair (e.g., biotin/streptavidin), or any other detectable moiety known to those skilled in the art.

The present disclosure is illustrated by the examples below.

### EXAMPLES

### Example 1: Materials and Methods

### Patients

NATIVE was a multicenter, randomized, placebo-controlled phase 2b study investigating the safety and efficacy of a treatment with lanifibranor in adult patients diagnosed with highly active, non-cirrhotic NASH. Patients were eligible for inclusion if they were 18 years of age or older and had non-cirrhotic NASH (the diagnosis of which required a Steatosis, Activity, Fibrosis [SAF] grade of 1 or higher for steatosis, hepatocellular ballooning and lobular inflammation on liver biopsy). A score of 3 or higher on the SAF-A (the activity part of the SAF scoring system that incorporates the scores for hepatocellular ballooning and lobular inflammation) was also a criterion for eligibility. Patients with stage F4 fibrosis, classified according to the criteria of both the SAF and NASH Clinical Research Network (NASH CRN) were excluded from the study.

Patients were randomly assigned in a 1:1:1 ratio to receive 800 or 1200 mg lanifibranor or placebo once daily for 24 weeks. Liver biopsy was obtained at baseline and at the end of treatment. The primary end point of the NATIVE study was defined as a decrease of at least 2 points from baseline to Week 24 (end of treatment) in the SAF-A score and no worsening of fibrosis. Secondary histologic end points included resolution of NASH (defined as a ballooning grade of 0 and a lobular inflammation grade of ≤1) and no worsening of fibrosis; improvement in fibrosis of at least 1 stage and no worsening of NASH (i.e., no worsening in either steatosis, ballooning, or lobular inflammation); Nonalcoholic Fatty Liver Disease Activity Score (NAS) improvement (defined as a decrease of ≥2 points from baseline to week 24 in the NAS) and no worsening of fibrosis; resolution of NASH and an improvement in fibrosis stage of at least 1 (as a composite end point); change in scores for the components of the SAF and NASH CRN scoring system (steatosis, activity, inflammation, ballooning, and fibrosis); and change in the modified Ishak score. Non-histologic secondary endpoints included changes in a panel of serum biomarkers related to metabolism, inflammation tissue injury and fibrosis.

Patients receiving lanifibranor 800 or 1200 mg per day were pooled and those with pre- and post-treatment liver biopsies were selected (n=142). Post-treatment biopsies were considered if they occurred within 14 days following last lanifibranor intake.

### Biomarkers

A total of 71 biomarkers of interest including 65 laboratory parameters and six diagnosis scores already published (FIB4, NAFLD fibrosis score (NFS), FIBC3, ABC3D, ELF and MACK3) were measured at baseline and at the end of treatment (EOT), i.e. at Week 24. The 65 laboratory biomarkers were mainly related to liver enzymes, lipid and glucose metabolisms, inflammation, and liver fibrosis, as shown in Table 1.

TABLE 1

| Category | Parameter name (unit) |
|---|---|
| Liver | Aspartate Aminotransferase (U/L) |
| enzymes | Alanine Aminotransferase (U/L) |
| | Gamma Glutamyl Transferase (U/L) |
| | Alkaline Phosphatase (U/L) |
| | Bilirubin (μmol/L) |
| Lipid | Cholesterol (mmol/L) |
| metabolism | HDL Cholesterol (mmol/L) |
| | LDL Cholesterol (mmol/L) |
| | Triglycerides (mmol/L) |
| | Apolipoprotein A1 (mg/dL) |
| | Apolipoprotein B (mg/dL) |
| | Apolipoprotein CIII (μg/mL) |
| | Adiponectin (μg/mL) |
| | Free Fatty Acid (mmol/L) |
| | Leptin (ng/mL) |
| | Fibroblast Growth Factor 21 (pg/mL) |
| Glucose | Glucose (mmol/L) |
| metabolism | Insulin (pmol/L) |
| | Insulin Resistance |
| | C-peptide (nmol/L) |
| | Free Fatty Acid (mmol/L) |
| | Fructosamine (μmol/L) |
| | Hemoglobin A1C (%) |
| | N-Terminal ProB-type Natriuretic Peptide (pmol/L) |
| Inflammatory | Fibrinogen (g/L) |
| markers | C-Reactive Protein (mg/L) - High Sensitivity |
| | Alpha-2 Macroglobulin (g/L) |
| | Haptoglobin (g/L) |
| | Interleukin 13 (pg/mL) - Not retained for analysis |
| | Interleukin 17 Alpha (pg/mL) |
| | Interleukin 1 Beta (pg/mL) |
| | Interleukin 6 (pg/mL) |
| | Interferon Gamma (pg/mL) |
| | Tumor Necrosis Factor (pg/mL) |
| Fibrosis | Hyaluronic Acid (ng/mL) |
| markers | Matrix Metalloproteinase 2 (%) |
| | Matrix Metalloproteinase 9 (%) |
| | Procollagen 3 N-Terminal Propeptide (ng/mL) |
| | N-Terminal Type 3 Collagen Propeptide (μg/L) |
| | Tissue Inhibitor of Metalloproteinase 1 (ng/mL) |
| | Tissue Inhibitor of Metalloproteinase 2 (ng/mL) |
| | Cytokeratin 18 Fragment M30 (pmol/L) |
| | Cytokeratin 18 Fragment M65 (pmol/L) |
| Other | Albumin (g/L) |
| biomarkers | Urea (mmol/L) |
| | Estimated Glomerular Filtration Rate (mL/min/1.73 m²) |
| | Prothrombin International Normalized Ratio |
| | Platelets (10⁹/L) |
| | Creatinine (μmol/L) |
| | Creatine Kinase (U/L) |
| | Basophils (10⁹/L) |
| | Eosinophils (10⁹/L) |
| | Hematocrit (L/L) |
| | Hemoglobin (g/L) |
| | Lymphocytes (10⁹/L) |
| | Mean Corpuscular Hemoglobin Concentration (g/L) |
| | Mean Corpuscular Volume (fL) |
| | Monocytes (10⁹/L) |
| | Neutrophils (10⁹/L) |
| | Erythrocytes (10¹²/L) |
| | Reticulocytes (10⁹/L) |
| | Ferritin (μg/L) |
| | Iron (μmol/L) |
| | Transferrin (g/L) |
| Published | ABC3D Score |
| diagnostic | ELF Score |
| Scores | FIB4 Score |
| | FIBC3 Score |
| | NAFLD Fibrosis Score |
| | MACK3 Score |

The accuracy of the six diagnostic scores for the prediction of treatment response was evaluated first. Then, the baseline value and the evolution under treatment of the 65 laboratory parameters were included to derive new combined biomarker signatures.

### Endpoint

The following histological endpoint according to NASH-CRN criteria was considered: NASH resolution without worsening of fibrosis (E2).

### Statistical Methods

### 1/Evaluation of the Available Diagnostic Scores

We evaluated the ability to assess the treatment response of the six published diagnostic scores available in the dataset: FIB4, NFS, FIBC3, ABC3D, ELF and MACK3. For each score, a multivariate model was derived including each the baseline value of the diagnostic score, its absolute change between baseline and EOT, and its relative change between baseline and EOT. The discriminatory ability of the models was assessed through the Area Under the Receiver Operating Characteristics (AUROC) curve. AUROC ranges from 0 to 1 and is interpreted as follows: 0.90-1.00=excellent, 0.80-0.90=good, 0.70-0.80=fair, and <0.70=poor discriminant abilities. Tests showing an AUROC≥0.8 are considered to be of clinical interest.

### 2/Development of the New Signatures for Treatment Response Assessment

Overall, biomarker selection was done using classical univariate analysis, Principal Component Analysis (PCA) and sparse Partial Least Square Discriminant Analysis (sPLS-DA), and finally combined in scores by logistic regression. The 65 biomarkers were considered in three different ways: baseline value, absolute and relative changes between baseline and EOT, leading to 195 variables considered for analysis.

### First Shortlisting

Univariate analysis using Student's t test, Welch's t test or Wilcoxon-Mann-Whitney test when appropriate was performed to identify the biomarkers significantly associated with the endpoint evaluated. Biomarkers with p-values below 10% were retained in the first shortlist. We additionally performed three runs of PCA and sPLS-DA: one including all baseline biomarkers, one including all absolute changes between baseline and EOT, and the last including all relative changes between baseline and EOT. For PCA and sPLS-DA analyses, Box-Cox and Yeo-Johnson transformations were applied to normalize biomarkers. PCA was used to evaluate the association between data variation and response status. In case of association, the best represented biomarkers on the PCA graphs (with cosine squared greater than 0.35) were selected. sPLS-DA aimed to identify discriminating biomarkers between responders and non-responders in a supervised way, and we retained the top 15 from this analysis. Finally, biomarkers selected by the seven analyses (univariate analysis, three PCA and three sPLS-DA) constituted the first shortlist of candidates for discrimination between responders and non-responders.

### Second Shortlisting

During this second phase, all candidate biomarkers from the first shortlist were introduced in a “final PCA” and a “final sPLS-DA”, finally leading to a second shortlist of up to 15 biomarkers. Correlation between biomarkers in this second shortlist was controlled, and only biomarkers with variance inflation factors lower than 4 were kept in the final biomarker shortlist.

### Logistic Regression Model

The study signature was constructed using logistic regression including the biomarkers of the second shortlist. Model selection was conducted through Akaike information criterion stepwise procedure while controlling for interactions. Finally, a regression formula was retrieved from the logistic regression to compute the probability of being a responder.

### 3/Evaluation of Accuracy of the Signatures

### Performance Evaluation

The discriminatory ability of the signature obtained for E2 endpoint was assessed through the AUROC. The calibration (statistical consistency between the predicted probability and the observations, i.e. predicted probabilities being on average close to 1 for responders and close to 0 for non-responders) was assessed through the Brier Score (BS). The BS ranges from 0 to 1, the lower the BS, the better the calibration, i.e. BS of 0 means perfect calibration

### Use in Clinical Practice

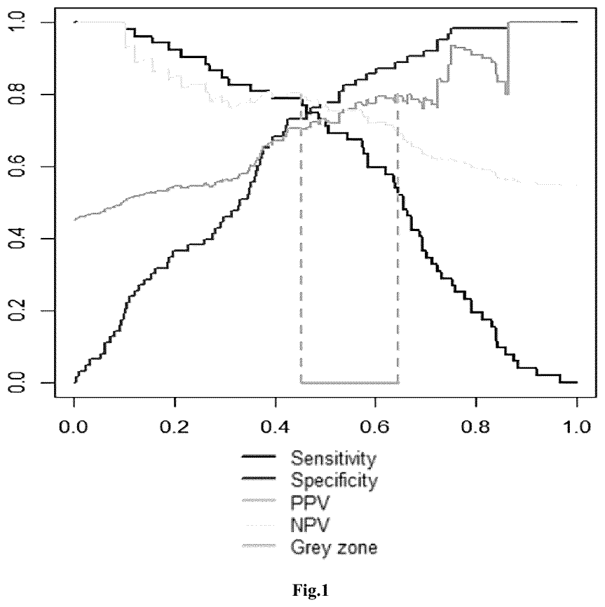

In clinical practice, a signature predicting the histological response based on non-invasive tests would be useful to assess lanifibranor treatment efficacy. Thresholds at 80% negative/positive predictive values (NPV/PPV) could be utilized to rule out/in treatment response, providing two cut-off values indicating the likelihood to be a non-responder (i.e. lower than the lowest cut-off value), a responder (i.e. higher than the highest cut-off value) or in the grey zone (i.e. between the 2 cut-off values). A practicable decision rule based on this approach is depicted in FIG. 1. We chose to calculate the thresholds for NPV/PPV because the assessment of treatment response is at the individual level, and not at the level of a population (for which sensitivity and specificity are better dedicated).

The diagnostic performance of the thresholds calculated was assessed through sensitivity, specificity, negative and positive predictive values, the size of the grey zone (the smaller, the better), and the non-invasive diagnostic effectiveness which is the rate of well classified patients among those outside the grey zone (the higher, the better).

### Software

R software was used, including the following packages: FactoMiner, MixOmics, CAR, stats, pROC, DescTools, ModelGood.

### Example 2: Results

### Study Population

In the NATIVE trial, from February 2017 through July 2019, 247 patients were randomly assigned to receive 1200 mg of lanifibranor (N=83), 800 mg of lanifibranor (N=83), or placebo (N=81) orally once daily for 6 months. A total of 228 patients completed the trial, 77 in each lanifibranor group and 74 in the placebo group. Reasons for discontinuation of the trial regimen have been reported elsewhere. Finally, among the 154 patients treated with lanifibranor at both doses, 142 patients (N=70 and N=72 patients treated with 800 or 1200 mg per day, respectively) presented with pre- and post-treatment liver biopsies within the time frame required for the present work and were included in the analysis population for signature development.

Demographic and clinical baseline characteristics for the whole population of 247 randomized and treated patients have been reported. A summary of baseline characteristics of the 142 patients retained in the analysis population for signature development is provided in table 2.

TABLE 2*

| Characteristic | Total population (N = 142) |
|---|---|
| Female sex - no. (%) | 89 (63) |
| Age - years | 54.6 ± 11.9 |
| Body-mass index - kg/m² | 32.8 ± 5.5 |
| Obese - no. (%) | 90 (63) |
| Type 2 diabetes mellitus - no. (%) | 60 (42) |
| Steatosis grade¹ | 2.57 ± 0.67 |
| Lobular inflammation grade² | 1.47 ± 0.50 |
| Ballooning grade³ | 1.77 ± 0.42 |
| Fibrosis stage⁴ | 2.09 ± 0.78 |
| Fibrosis stage F2 or F3 - no. (%) | 111 (78%) |
| SAF-A score⁵ | 3.25 ± 0.46 |
| NAS⁶ | 5.89 ± 0.96 |
| NAS ≥ 6 - no. (%) | 104 (73) |
| Alanine aminotransferase - IU/L | 63.3 ± 42.2 |
| Aspartate aminotransferase - IU/L | 48.9 ± 35.4 |
| Gamma Glutamyl transferase - IU/L | 79.3 ± 112 |
| Fasting HDL cholesterol - mmol/L | 1.23 ± 0.3 |
| Fasting triglycerides - mmol/L | 1.92 ± 0.87 |
| Fasting glucose - mmol/L | 6.08 ± 1.51 |
| Hemoglobin A1C - % | 6.09 ± 0.76 |
| Fasting insulin level - pmol/L | 257 ± 272 |
| Fructosamine - μmol/L | 245 ± 29.8 |
| Adiponectin - μg/mL | 5.22 ± 3.3 |
| Ferritin - μg/L | 242 ± 242 |
| Transferrin - g/L | 2.88 ± 0.47 |
| MMP-9 - % | 57.4 ± 44.9 |
| Hyaluronic acid - ng/mL | 90.8 ± 123 |
| Cytokeratin 18 Fragment M65 - pmol/L | 845 ± 960 |
| ABC3D⁷ | 2.84 ± 1.53 |
| FIBC3⁸ | −0.89 ± 1.53 |
| FIB4⁹ | 1.45 ± 0.88 |
| MACK3¹⁰ | 0.51 ± 0.28 |
| ELF¹¹ | 9.74 ± 1.04 |
| NAFLD fibrosis score¹² | −1.36 ± 1.39 |

*Plus-minus signs are means ± standard deviation. To convert the values for high-density lipoprotein (HDL) cholesterol to milligrams per deciliter, divide by 0.02586. To convert the values for triglycerides to milligrams per deciliter, divide by 0.01129. To convert the values for glucose to milli- grams per deciliter, divide by 0.01129. To convert the values for insulin to micrograms per liter, divide by 172.2.
¹Steatosis was assessed as the percentage of hepatocytes containing large and medium-sized intracytoplasmic lipid droplets (but not foamy microvesicles) and graded as 0 (<5%), 1 (5 to 33%), 2 (34 to 66%), or 3 (≥67%), according to the Steatosis, Activity, Fibrosis (SAF) scoring system. Patients with grade 0 steatosis were excluded from the trial.
²Lobular inflammation was classified as grade 1 (two small foci of inflammatory cells) or grade 2 (more than two foci of inflammatory cells), according to the SAF scoring system.
³Ballooning was classified as grade 1 (round hepatocytes with pale cytoplasm and size similar to that of normal hepatocytes) or grade 2 (presence of enlarged hepatocytes with a diameter at least twice that of normal hepatocytes in a background of clear and round hepatocytes), according to the Nonalcoholic Steatohepatitis Clinical Research Network (NASH CRN) grading system.
⁴Fibrosis was classified as stage F0 (no fibrosis), stage F1 (mild fibrosis), stage F2 (significant [moderate] fibrosis), stage F3 (advanced fibrosis), or stage F4 (cirrhosis), according to the SAF-NASH CRN staging system. Patients with stage F4 fibrosis were excluded from the trial.
⁵The SAF-Activity (SAF-A) score ranges from 0 to 4; with higher scores indicating more-severe disease activity.
⁶The Nonalcoholic Fatty Liver Disease Activity Score (NAS) ranges from 0 to 8. A score of 2 or less indicates “not NASH”; a score of 3 or 4, “borderline NASH,” and a score of 5 to 8, “definite NASH.”
⁷1 The ABC3D score comprises: A = Age > 50 years, B = BMI > 30 kg/m², C = platelet Count <200 × 10\hat{ }9/L, 3 = PRO-C3 > 15.5 ng/ml, D = Diabetes = present. The presence of each factor scores 1 point, except for T2DM that scores 2 points, yielding to a maximum of 6 (See Boyle M et al,. “Performance of the PRO-C3 collagen neo-epitope biomarker in non-alcoholic fatty liver disease”, JHEP Rep. 2019; 1(3): 188-198)
⁸2 FIBC3 score is calculated using the following formula: −5.939 + (0.053*Age in years) + (0.076*BMI in kg/m²) + (1.614*T2DM (yes = 1, no = 0)) − (0.009*platelets in 10\hat{ }9/L) + (0.071*PRO-C3 in ng/ml) (See Boyle M et al,. “Performance of the PRO-C3 collagen neo-epitope biomarker in non-alcoholic fatty liver disease”, JHEP Rep. 2019; 1(3): 188-198)
⁹3 FIB4 score is calculated using the following formula: (age (years) * AST (U/L))/(platelets (×10⁹/L) * √ALT (U/L)) (See Sterling RK, et al. “Development of a simple noninvasive index to predict significant fibrosis in patients with HIV/HCV coinfection”. Hepatology 2006; 43(6): 1317-25)
¹⁰4 MACK3 score is based on the following parameters: AST (IU/L), Glycemia (mmol/L), Insulin (μU/mL) and Cytokeratin M30 (IU/L) (see Boursier J et al., “Screening for therapeutic trials and treatment indication in clinical practice: MACK-3, a new blood test for the diagnosis of fibrotic NASH. Aliment”, Pharmacol Ther. 2018; 47(10): 1387-9).
¹¹5 ELF score is calculated using the following formula: 2.494 + 0.846 ln(Hyaluronic acid) + 0.735 ln(PIIINP) + 0.391 ln(TIMP-1) (See Lichtinghagen R et al. “The Enhanced Liver Fibrosis (ELF) score: normal values, influence factors and proposed cut-off values”, J Hepatol. 2013; 59(2): 236-42)
¹²6 NAFLD Fibrosis Score is calculated using the following formula: −1.675 + 0.037 * age (years) + 0.094 * BMI (kg/m²) + 1.13 * (Impaired Fasting Glucose/diabetes (yes = 1, no = 0)) + 0.99 * (AST/ALT ratio (no unit)) − 0.013*platelets (×10⁹/L) − 0.66*albumin (g/dl) (See Angulo Pet al. “The NAFLD fibrosis score: a non-invasive system that identifies liver fibrosis in patients with NAFLD”, Hepatology 2007; 45(4): 846-54)

The mean age of the patients was 55 years, and the mean body mass index (BMI) was 33; 89 patients (63%) were female, and 60 (42%) had type 2 diabetes mellitus. Significant or advanced fibrosis (stages F2 and F3, respectively) was present in 111 patients (78%), and most patients had highly active NASH (the mean [±SD] SAF-A score was 3.3±0.5, and 73% had a NAS of ≥6, which indicates high disease activity).

Resolution of NASH without worsening of liver fibrosis (E2) was demonstrated in 63 patients (44%) [27 patients (39%) treated with 800 mg and in 36 patients (50%) treated with 1200 mg].

### Available Diagnostic Scores

In a first step, the performance of currently available diagnostic scores FIB4, FIBC3, ABC3D, NFS, ELF and MACK3 in predicting histological responders with regard to the endpoint E2 was assessed. For the prediction of E2, MACK-3 model (baseline value combined with absolute change) provided the highest AUROC at 0.78. ELF provided the only other model combining baseline value with evolution under treatment, with AUROC at 0.68.

The accuracy of published diagnostic score for the prediction of E2 is presented in table 3.

TABLE 3

E2

| | Biomarkers included | |
|---|---|---|
| Scores | in the model | AUROC |
| MACK3 | Baseline raw values + | 0.78 |
| | absolute changes at EOT | |
| FIBC3 | Absolute changes at EOT | 0.62 |
| NFS | Absolute changes at EOT | 0.62 |
| ELF | Absolute + relative | 0.68 |
| | changes at EOT | |
| FIB4 | NONE | — |
| ABC3D | NONE | — |

None of the models reached AUROC at 0.80 for E2, indicating that existing scores are not sufficiently accurate to predict these endpoints. Therefore, further analyses focused on the development of new and specific signature for E2.

### Example 3

Development of a Specific Non-Invasive Biomarker Signature for E2 (NASH Resolution without Worsening of Fibrosis)

The signature was built considering the 65 laboratory biomarkers available in the study, in their three different ways (baseline raw values, absolute changes at EOT and relative changes at EOT), providing a total of 195 biomarkers. Of these, 11 biomarkers were selected (*) for the final shortlist (see Table 4).

TABLE 4

| | Baseline | Absolute | Relative |
|---|---|---|---|
| | raw | changes at | changes at |
| | values | Week 24 | Week 24 |
| AST | X | X | X |
| ALT | X | | X* |
| Ferritin | X | X | |
| C-peptide | X | X | X |
| Insulin | X | X | |
| BNPPRONT | X | X | |
| CK18M30 | X | | |
| CK18M65 | X* | | |
| PROC3 | X | X | X |
| Hemoglobin | X | | X |
| MCHC | X | X* | X |
| Erythrocytes | X | | |
| ALP | X | X | X |
| Adiponectin | | X | X |
| Fructosamine | | X | X* |
| Transferrin | | X | X |
| Hyaluronic acid | | X* | |
| MMP2 | | X | X* |
| Eosinophils | | X | |
| Hematocrit | X | X | X |
| MCV | | X | X |
| Neutrophils | | X | X |
| Leukocytes | X* | X | X |
| GGT | X | X | X* |
| MMP9 | | | X |
| P3NP | X | X | |
| TIMP1 | X | X | |
| Iron | X | | |
| Leptin | X | | |
| Bilirubin | X | | |
| Interferon gamma | X* | X | |
| Free fatty acid | X* | | |
| Albumin | X* | | |
| Creatine kinase | | X | |
| Apolipoprotein CIII | | X | |
| Glucose | | | X |

Among these 11 biomarkers, 4 were finally selected by logistic regression as independent predictors of E2: baseline CK18M65, absolute change of hyaluronic acid, relative change of fructosamine and relative change of ALT. Combining these 4 parameters into an E2-score provided AUROC of 0.80±0.08 for predicting E2 response. Calibration of E2 was good with a Brier score at 0.18 (see Table 5).

TABLE 5

| | E2-score |
|---|---|
| AUROC | 0.80 ± 0.08 |
| Brier Score | 0.18 |
| 80% negative predictive | 0.453 |
| value threshold | |
| Rate of patients in | 50% |
| refusal zone (%) | |
| Sensitivity (%) | 79% |
| 80% positive predictive | 0.646 |
| value threshold | |
| Rate of patients in | 30% |
| acceptation zone (%) | |
| Sensitivity (%) | 89% |
| Rate of patients in | 20% |
| grey zone (%) | |
| Non-invasive diagnostic | 80% |
| effectiveness | |

Sensitivity (descending curve, top left to bottom right), specificity (ascending curve, bottom left to top right), NPV (descending curve, top left to middle right) and PPV (ascending curve, middle left to top right) curves as a function of E2-score results are depicted in FIG. 1. The predictive value thresholds were 0.453 (80% negative predictive value with corresponding 79% sensitivity) and 0.646 (80% positive predictive value with corresponding 89% specificity). Using these thresholds, 80% of patients were identified as biomarker E2 non-responders or responders, with 80% of them confirmed histologically, leaving only 20% patients in the grey zone.

These results suggested that the retrieved biomarker signature consisting of CK18M65, hyaluronic acid, fructosamine and ALT can be considered as a good classifier of endpoint E2.

It thus results that the specific combination of biomarkers (CK18M65, hyaluronic acid, fructosamine and ALT) allowed the assessing the effectiveness of a treatment with lanifibranor in non-cirrhotic NASH, with good diagnostic performance in particular for NASH resolution without worsening of fibrosis.

Aspects of the present disclosure are further illustrated by reference to the following, non-limiting embodiments.
1. A method of assessing the effectiveness of a treatment with lanifibranor in a patient with a liver disease, the method comprising:
   a) an in vitro measurement of a level of a combination of biomarkers consisting of CK18M65, hyaluronic acid, fructosamine and ALT in a biological sample from the patient, and
   b) an assessment of the effectiveness of a treatment with lanifibranor in the patient as a function of the level measured for the combination of biomarkers.
2. A method of assessing the effectiveness of a treatment with lanifibranor in a patient with a liver disease, the method comprising:
   a1) an in vitro measurement of a level of a combination of biomarkers consisting of CK18M65, hyaluronic acid, fructosamine and ALT in a biological sample from the patient;
   b1) a comparison of the level measured in step a) compared to that measured in a plurality of samples of patients with a liver disease and having received a treatment with lanifibranor for which the effectiveness of treatment is known; the comparison being carried out by means of a statistical learning model using as input data the levels of the combination of biomarkers measured at step a); and
   c1) an assessment of the effectiveness of a treatment with lanifibranor in the patient as a function of the results determined by the model defined at step b1).
3. The method of paragraph 1 or 2, wherein the level of CK18M65 is measured before the start of the treatment with lanifibranor and the levels of hyaluronic acid, fructosamine and ALT are measured before the start of the treatment and after at least 3 months of treatment with lanifibranor.
4. The method of paragraph 3, wherein the levels of the combination of biomarkers measured in step a1) are used to obtain a score linked to the assessment of the effectiveness of the treatment in the patient, the score being compared with at least one predetermined cutoff value so as to classify the prognosis among a plurality of classes.
5. The method of paragraph 4, wherein the plurality of classes comprises at least two classes of which one class of non-response to the treatment with lanifibranor.
6. The method of paragraph 4 or 5, wherein the assessment of the effectiveness of the treatment in the patient comprises a comparison of the score with a first calculated cutoff value below which poor or no effectiveness can be predicted and a second calculated cutoff value above which good effectiveness can be predicted.
7. The method of any of paragraphs 2 to 6, wherein the learning model is based on a prior analysis of samples of a cohort comprising patients treated with lanifibranor presenting a good response to the treatment and patients treated with lanifibranor presenting poor or no response to the treatment.
8. The method of paragraph 7, wherein said prior analysis comprises the application of a method for learning and for selecting variables.
9. The method of paragraph 8, wherein said method for learning and for selecting variables is a logistic regression.
10. The method of any of paragraphs 7 to 9, wherein the levels are weighted as a function of the prior analysis of said cohort to derive the score.
11. The method of paragraph 1 or 2, wherein the biological sample is a sample of biological fluid.
12. The method of paragraph 11, wherein the sample of biological fluid is a sample of blood, serum or plasma.
13. The method of paragraph 1 or 2, wherein the biomarker(s) the level of which is measured in step a) is/are protein biomarker(s).
14. The method of paragraph 1 or 2, wherein the liver disease is non-alcoholic fatty liver disease, non-alcoholic steatohepatitis, compensated or decompensated cirrhosis, liver fibrosis, liver fibrosis, fatty liver disease, acute liver failure or acute-on-chronic liver failure.
15. A system for assessing the effectiveness of a treatment with lanifibranor in a patient with a liver disease, the system comprising:
   a) means for measuring or receiving measurement data of a level of a combination of biomarkers consisting of CK18M65, hyaluronic acid, fructosamine and ALT in a biological sample from the patient; and
   b) means for processing measurement data configured to assess an effectiveness of the treatment with lanifibranor in the patient as a function of the level measured for the combination of biomarkers.
16. A combination of biomarkers consisting of CK18M65, hyaluronic acid, fructosamine and ALT for use in a method of assessing the effectiveness of a treatment with lanifibranor in a patient with a liver disease.

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, composition of matter, means, methods and steps described in the specification. As one of ordinary skill in the art will readily appreciate from the disclosure of the presently disclosed subject matter, processes, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, compositions of matter, means, methods, or steps.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the device, method, and system of the disclosed subject matter without departing from the spirit or scope of the disclosed subject matter. Thus, it is intended that the disclosed subject matter include modifications and variations that are within the scope of the appended claims and their equivalents.

For any patents, patent applications, publications, product descriptions, and protocols are cited throughout this application, the disclosures of all of which are incorporated herein by reference in their entireties for all purposes.

1. A method of assessing the effectiveness of a treatment with lanifibranor in a patient with a liver disease, the method comprising:
a) in vitro measuring levels of a combination of biomarkers consisting of CK18M65, hyaluronic acid, fructosamine and ALT in a biological sample from the patient; and
b) assessing the effectiveness of the treatment with lanifibranor as a function of the levels measured in step a).

2. The method of claim 1, wherein the expression level of CK18M65 is measured before the start of the treatment with lanifibranor.

3. The method of claim 1, wherein the levels of hyaluronic acid, fructosamine and ALT are measured before the start of the treatment with lanifibranor and after at least 3 months of treatment with lanifibranor.

4. The method of claim 1, wherein the levels measured in step a) are used to obtain a score which falls within a determined or undetermined prognosis class.

5. The method of claim 4, wherein the score is obtained by combining the levels measured in step a) in a mathematical function.

6. The method of claim 5, wherein the mathematical function is a binary logistic regression.

7. The method of claim 4, which comprises comparing the score with a first calculated cutoff value below which no response to the lanifibranor treatment can be predicted.

8. The method of claim 4, which comprises comparing the score with a second calculated cutoff value above which a response to the lanifibranor treatment can be predicted.

9. The method of claim 1, wherein the biological sample is a sample of biological fluid.

10. The method of claim 9, wherein the biological fluid is blood, serum or plasma.

11. The method of claim 1, wherein the liver disease is non-alcoholic fatty liver disease, non-alcoholic steatohepatitis, compensated or decompensated cirrhosis, liver fibrosis, fatty liver disease, acute liver failure or acute-on-chronic liver failure.

12. A system for assessing the effectiveness of a treatment with lanifibranor in a patient with a liver disease, the system comprising:
a) a means for measuring or receiving measurement data of levels of a combination of biomarkers consisting of CK18M65, hyaluronic acid, fructosamine and ALT in a biological sample from the patient; and
b) a means for processing the data configured to assess the effectiveness of the treatment with lanifibranor in the patient as a function of the levels measured for the combination of biomarkers.

13. A combination of biomarkers consisting of CK18M65, hyaluronic acid, fructosamine and ALT for use in a method of assessing the effectiveness of a treatment with lanifibranor in a patient with a liver disease.

14. The combination of claim 13, wherein the liver disease is non-alcoholic fatty liver disease, non-alcoholic steatohepatitis, compensated or decompensated cirrhosis, liver fibrosis, fatty liver disease, acute liver failure or acute-on-chronic liver failure.

15. A method of treating a liver disease in a subject, the method comprising:
a) in vitro measuring levels of a combination of biomarkers consisting of CK18M65, hyaluronic acid, fructosamine and ALT in a biological sample from the subject before initiating a treatment;
b) daily administering an effective amount of lanifibranor to the subject for at least 3 months;
c) in vitro measuring levels of hyaluronic acid, fructosamine and ALT in a biological sample of the subject after 3 months of treatment;
d) assessing the effectiveness of the treatment with lanifibranor as a function of the levels measured in steps a) and c), whereby a response or a non-response to the lanifibranor treatment can be predicted; and
e) continuing administering an effective amount of lanifibranor to the subject for at least another 3 months provided the assessment made in step d) is predictive of a response to the lanifibranor treatment, or that no diagnostic can be made.

16. The method of claim 15, wherein the assessment in step d) is made by obtaining a score from the levels measured in steps a) and c), and comparing the score (i) with a first calculated cutoff value below which no response to the lanifibranor treatment can be predicted, and (ii) with a second calculated cutoff value above which a response to the lanifibranor treatment can be predicted.

17. The method of claim 16, wherein resolution of NASH and no worsening of liver fibrosis can be predicted when the score obtained is above the second calculated cutoff value.

18. The method of claim 15, wherein the biological sample is a sample of biological fluid.

19. The method of claim 18, wherein the biological fluid is blood, serum or plasma.

20. The method of claim 15, wherein the liver disease is non-alcoholic fatty liver disease, non-alcoholic steatohepatitis, compensated or decompensated cirrhosis, liver fibrosis, fatty liver disease, acute liver failure or acute-on-chronic liver failure.
